(19) United States
(12) Patent Application Publication
(10) Pub. No.: US 2005/0121086 A1
(43) Pub. Date: Jun. 9, 2005

(54) Ventilator arrangement

(76) Inventor: Nils Olof Eriksson, Enskede (SE)

Correspondence Address: BANNER & WITCOFF, 1001 G STREET N W, SUITE 1100, WASHINGTON, DC, 20001, US

(21) Appl. No.: 10/501,534

(22) PCT Filed: Dec. 16, 2002
(86) PCT No.: PCT/SE02/02337

(30) Foreign Application Priority Data
Jan. 18, 2002 (SE) 0200129-5

(51) Int. Cl. A61M 16/20; F16K 11/22

(52) U.S. Cl. 137/625.4

(57) ABSTRACT

The invention comprises a valve arrangement (10) with at least two inlets (20, 21), adapted each for a medium (A, B), and at least one outlet (22), through which a chosen mixture of the said media can pass. The said inlets (20, 21) are adapted to interact each via a section (20a, 21a) of channel with associated openings (12c, 12b) in a valve seating (12) while the said outlet (22) is adapted to interact via a section (22a) of channel with an associated opening (22) in a valve gate (11). A first means (30) is available in order to control at least the motion of the valve gate (11) relative to the valve seating (12) that at least one chosen mixing ratio and/or one chosen rate of flow can thus be regulated. A glide surface and/or an interface (11a, 12a) between the valve seating (12) and the valve gate (11) is chosen to be plane, or at least essentially plane. A further means (40) is available in order to control the motion of the valve seating (12) relative to the valve gate (11) in such a manner that a chosen rate of flow can be regulated. The said two means (30, 40) for controlling the motion of the valve gate (11) and the motion of the valve seating (12) are formed to comprise two different devices (31, 41) each controlling one motion.

### TECHNICAL AREA

The present invention relates in general to a valve arrangement and in particular to such a valve arrangement to which has been assigned at least two inlets, each adapted for one medium, and at least one outlet, through which a chosen mixture of the said media can flow.

More specifically, the present invention relates to such valve arrangements in which the said inlets are adapted to be able to interact each via a section of channel with the associated openings in a valve seating while the said outlet is adapted to interact via a section of channel with an associated opening in a valve gate or vice versa.

A first means is available in order to control the movement of the valve gate relative to the valve seating or vice versa at least in such a manner that it is possible to regulate, at least, a chosen mixture ratio and/or a chosen rate of flow.

Valve arrangements with the features described in the introduction are described as “multiseated valves”.

Valve arrangements or multiseated valves of this type can be used when it is a question of being able to offer flow regulation of several media, in fluid form or in gas form.

Multiseated valves of this type are normally constructed with a casing and with at least two valve seats formed in the casing, together with a valve body or valve gate contained by the casing and arranged to be displaceable, with surface sections that face the valve seatings and that are adapted to be able in a first end position of the valve body within the casing to make contact with the said valve seatings and to form a seal, and to be able in a second end position of the valve body to create a passage of the said media through the valve seatings and the said surface sections.

It is clear that it must be possible for the said valve body or valve gate to take one or several intermediate positions between the said end positions in order in this way to be able to create a regulated passage of the said media, such as a flow regulation.

The invention has principally been developed to be able to regulate the rate of flow under chosen mixing conditions of air and oxygen in pre-determined proportions, and it aims at achieving application for lung ventilator equipment when these are used for patients when under the influence of intravenous anaesthesia.

### PRIOR ART

Methods and arrangements associated with multiseated valves, such as multiseated valves for control of a rate of flow and/or the mixing of several media, with the features specified above and for the application described above are previously known in a number of different embodiments.

As a first example of the prior art, such valves as those intended to mix a number of media, present in gas form, with a casing and at least two valve seats can be mentioned.

Valve arrangements and mixing valves of this type have been provided with one valve body, contained within the casing and arranged to be axially displaceable via a rotary motion, with two surface sections that face two valve seatings, and which surface sections belonging to the valve body are adapted to be able to make contact at a first end position of the rotational movement of the valve body with and to press against one of the said valve seatings while the second valve seating is open and at a second end position of the rotational movement of the valve body to create an opening for the first valve seating and to allow the valve body to make contact with, in a sealing manner and to press against, the second valve seating and in at least one of these end positions to offer a relatively free passage of the said media though one of the valve seatings and the said surface section.

This embodiment will offer for each intermediate position a mixing ratio that corresponds to the intermediate position of the valve body.

A mixing valve of this type is revealed and described in more detail in the international patent document PCT/SE00/01067.

Furthermore, for this embodiment of the casing it has been proposed that the said casing offers a first inlet for a first medium and a first channel, in order to connect the said first inlet with the said first valve seating, and a second inlet for a second medium and a second channel, in order to connect the said second inlet with the said second valve seating.

The valve seatings are furthermore coordinated with one or two channels internal to the casing in order to connect to one or two inlets belonging to the casing for each of the said media in order to obtain mixing of these media downstream of the casing.

The actual mixing process can also be carried out within the casing of the mixer valve in a cavity formed therein.

### DESCRIPTION OF THE PRESENT INVENTION

Technical Problems

If the situation is considered that the technical evaluations that one skilled in the arts must carry out within the relevant technical area in order to be able to offer a solution to one or several of the technical problems that are posed are not only an initial necessary insight into the measures and/or the sequence of measures that are to be taken, but also a necessary choice of the means (singular or plural) that are required, then the following technical problems should be, with respect to this, relevant during the development of the present invention.

When considering the prior art, as has been described above, therefore, it should be possible to regard as a technical problem the ability to realise the significance of and the advantages associated with offering a design for an arrangement in the form of a multiseated valve that can be used for an accurate and stable control of the rate of flow and/or a mixture of media, and proposing a simple method for being able to assemble and construct such a multiseated valve, such that the valve will be easy to assemble and can be constructed from a few simple parts.

Thus there lies a technical problem in the ability to realise the significance of, and the advantages associated with, using, for a valve arrangement with the features described in the introduction, the conditions present such that a contact surface and/or an interface between a valve seating and a valve gate is to be plane, or essentially plane.

Thus there lies a technical problem in the ability to realise the significance of, and the advantages associated with, in addition to a first means, at least controlling the motion of the valve gate relative to the valve seating in such a manner that a chosen input mixing ratio or input rate of flow can be regulated, to be able to create simple conditions for the use of a further means, in order to control the motion of the valve seating relative to the valve gate to thus control the input of a chosen rate of flow.

Thus there lies a technical problem in the ability to realise the significance of, and the advantages associated with, allowing two means to be formed to comprise two different devices, each controlling one of two motions, in order to control the motion of the valve gate and/or the motion of the valve seating.

Thus there lies a technical problem in the ability to realise the significance of, and the advantages associated with, allowing a first of these devices to be adapted for a first direction of motion and a second of these devices to be adapted for a second direction of motion.

Thus there lies a technical problem in the ability to realise the significance of, and the advantages associated with, allowing the first direction of motion to be adapted such that it exclusively, or at least to a dominating degree, controls the mixing ratio between the media, while the second direction of motion is to be able to be adapted to exclusively, or at least to a dominating degree, control the rate of flow of the medium passing through the outlet.

There lies a further technical problem in the ability to realise the significance of, and the advantages associated with, allowing a first device, which controls the motion of the valve gate, to be adapted to bestow upon the valve gate, via a rotational motion, a translation or displacement motion oriented along the direction of the interface.

Thus there lies a technical problem in the ability to realise the significance of, and the advantages associated with, allowing a second device, which controls the motion of the valve seating, to be adapted to bestow upon the valve seating, via a rotational motion, a translation or displacement motion oriented along the direction of the interface, where it is to be possible to choose the direction of this motion and/or the speed of the displacement distinct from the direction of motion and/or the speed of displacement offered by the first device.

Thus there lies a technical problem in the ability to realise the significance of, and the advantages associated with, allowing the directions of motion to be chosen straight and distinct or at an angle from each other, preferably at an angle of approximately 90°.

There also lies a technical problem in the ability to realise the significance of, and the advantages associated with, proposing a valve arrangement with the features specified in the introduction, which it is to be possible to use as a multiseated mixing valve for the mixing of a number of compressible media, used in a lung ventilator unit, in particular a lung ventilating unit in association with intravenous treatment, assigned to anaesthesia, and in this way to realise that the said media should be supplied to the said inlets at the same pressure, or at least at essentially the same pressure, whereby control of the rate of flow, via further means and the devices associated with it, can be carried out without influencing the mixing ratio, or control of the mixing ratio via further means and the devices associated with it, can be carried out without influencing the rate of flow.

Thus there lies a technical problem in the ability to realise the significance of, and the advantages associated with, allowing the gearing between the chosen turning motion/displacement ratio to be adapted to be equivalent to: one revolution=0.5-2.0 mm, for example 0.6-1.2 mm, and furthermore, to adapt the relationship differently for the different directions of the displacement motions.

Thus there lies a technical problem in the ability to realise the significance of, and the advantages associated with, allowing the shaped openings in the valve seating to be arranged beside each other and at a separation that corresponds to the length chosen for an opening in the valve gate.

Thus there lies a technical problem in the ability to realise the significance of, and the advantages associated with, allowing the openings in the valve seating within the interface to be chosen to be right-angled, with a width that corresponds to the total displacement motion for the chosen limiting values for the chosen mixing ratio, and with a length that corresponds to the total displacement motion for the chosen limiting values for the chosen control of rate of flow.

Thus there lies a technical problem in the ability to realise the significance of, and the advantages associated with, allowing the openings in the valve seating within the interface to be chosen with a narrowing or a widening width within the said length-related distance.

Thus there lies a technical problem in the ability to realise the significance of, and the advantages associated with, allowing the opening within the valve gate within the interface, and at least within the openings in the valve seating, to be chosen with a narrowing or a widening width.

It should probably also be seen as a technical problem to be able to realise the significance of, and the advantages associated with, creating the conditions required such that it will be possible to be press the valve gate against the valve seating with the aid of a spring unit and in this way to create an airtight, or at least essentially airtight, interface.

There also lies a technical problem in the ability to realise the significance of, and the advantages associated with, allowing the first direction of motion be offered via a turning motion at an angle, whereby the two displacement motions required can take place each via one of two axes of rotation oriented parallel to each other.

There lies also a technical problem in the ability to realise the significance of, and the advantages associated with, allowing the valve seating to be arranged to be displaceable in a backwards and forwards motion within a box-shaped first casing.

There lies also a technical problem in the ability to realise the significance of, and the advantages associated with, allowing the valve gate to be arranged to be displaceable in a backwards and forwards motion within a box-shaped second casing.

There lies also a technical problem in the ability to realise the significance of, and the advantages associated with, allowing a rotational motion to interact with an item that functions as a nut, while interacting in a somewhat mobile manner with the valve gate.

There lies also a technical problem in the ability to realise the significance of, and the advantages associated with, allowing a chosen ratio, between an open surface area for the first opening in the valve seating and the opening in the valve gate, and an open surface area for the second opening in the valve seating and the opening in the valve gate, to be adapted to offer a chosen mixing ratio.

There lies also a technical problem in the ability to realise the significance of, and the advantages associated with, allowing the sum of an open surface area for the first opening in the valve seating and the opening in the valve gate, and an open surface area for the second opening in the valve seating and the opening in the valve gate, to be adapted in order to offer a chosen rate of flow.

The Solution

The present invention thus is based on the prior art for a valve arrangement proposed in the introduction, in the form of a multiseated valve, and which is to serve as a mixing valve and where the valve arrangement is to offer at least two inlets, adapted each for one medium, and at least one outlet, thorough which a chosen mixture of the said media can flow, furthermore the said inlets are adapted to interact each via a section of channel with associated openings in a valve seating, while the said outlet is to be adapted to interact via a section of channel with an associated opening in a valve gate.

The valve arrangement is to use a first means in order to control the motion of the valve gate relative to the valve seating or vice versa at least such that at least one chosen mixing ratio and/or one chosen rate of flow can be adjusted.

In order to be able to solve one or several of the technical problems described above, the present invention proposes in particular the supplementation of the prior art with a glide surface and/or an interface between the valve seating while the valve seating and the valve gate are to be plane, or at least essentially plane, whereby it should be possible to use a further means to control the motion of the valve gate relative to the valve seating or vice versa.

A chosen rate of flow can in this way be adjusted, whereby the said two means, in order to control the motion of the valve gate and/or the motion of the valve seating, may be designed to comprise two different devices each controlling the respective motion.

As suggested embodiments falling within the scope of the basic concept of the present invention, it is proposed that it is to be possible for a first device to be adapted for a first direction of motion and it is to be possible for a second device to be adapted for a second direction of motion, and that the first direction of motion is adapted to exclusively, or at least to a dominant degree, control the mixing ratio between the media, while the second direction of motion is adapted to exclusively, or at least to a dominant degree, to control the rate of flow of the mixed media passing through the outlet.

Furthermore, the invention proposes that the first device, which controls the motion of the valve gate, is to be adapted such that it can bestow upon the valve seating via a rotational motion a translation or displacement motion oriented along the direction of the interface.

Furthermore, it is proposed that it is to be possible to adapt the second device, which controls the motion of the valve seating, such that it bestows upon the valve seating via a rotational motion a translation or displacement motion oriented along the direction of the interface, where it is to be possible to choose the direction of motion and/or the speed of displacement of this distinct from the direction of motion and/or the speed of displacement offered via the first device.

The invention in particular proposes that the directions of motion are to be chosen to be straight and distinct from each other, preferably at an angle of 90°.

In particular, the present invention proposes that for a valve arrangement of multiseated type, serving as a mixing valve, with the features described in the introduction, and where this valve arrangement is to be used for the mixing of a number of compressible media in a lung ventilator unit, in particular, a lung ventilator unit in association with intravenous treatment assigned to anaesthesia, that the said media are to be presented at the said inlets at the same pressure, or at least at essentially the same pressure, whereby regulation of the rate of flow, via the additional means and the device associated with it, can be carried out without influencing the mixing ratio, or an adjustment of the mixing ratio can be carried out, via the said means and the device associated with it, without influencing the rate of flow.

In particular, the present invention proposes for the application described above that a chosen gear ratio between the chosen turning motion/displacement motion is to be adapted such that it corresponds to one revolution=0.5-2.0 mm, for example 0.6-1.2 mm.

Furthermore, the invention proposes that the openings formed in the valve seating are to be arranged side-by-side at a distance that corresponds to a length selected for the opening in the valve gate.

Furthermore, the invention proposes that the openings in the valve seating are chosen to be right-angled in the interface, with a width that corresponds to the total displacement motion for the said limiting values for the chosen mixing ratio and with a length that corresponds to the total displacement motion for the chosen limiting values for the chosen regulation of the rate of flow.

Further, it is proposed that the openings in the valve seating are, within the interface, chosen with a narrowing or with a widening width within the said distance related to length.

The possibility of allowing the opening in the valve gate to be chosen, within the interface and at least within the openings in the valve seating, with a narrowing or with a widening width also falls within the scope of the invention.

The invention also proposes that it is to be possible, with the aid of a spring unit, to press the valve gate against the valve seating and in this way to form an airtight interface between the valve gate and the valve seating with the aid of lubricating means used there.

The invention also proposes that it is to be possible to offer the first direction of motion via a turning motion at an angle, whereby the two displacement motions related by an angle can take place each via its own rotational motion assigned to each one of two axes of rotation oriented parallel to each other.

The invention also proposes that it is to be possible to arrange the valve seating to be displaceable in a backwards and forwards motion within a box-shaped first casing, while it should be possible to arrange the valve gate to be displaceable forwards and backwards within a box-shaped second casing.

The invention also proposes to allow each of the said rotational motions, or at least the one assigned to the gate, to be allowed to interact with a nut, while interacting in a somewhat mobile manner with the valve gate and/or valve seating.

The invention also proposes that it is to be possible to adapt a chosen ratio, between an open surface area for the first opening in the valve seating and the opening in the valve gate, and an open surface area for the second opening in the valve seating and the opening in the valve gate such that it offers a chosen mixing ratio.

The sum of an open surface area for the first opening in the valve seating and the opening in the valve gate and an open surface area of the second opening in the valve seating and the opening in the valve gate, is adapted in order to offer a chosen rate of flow.

Advantages

The advantages that can be principally regarded as characteristic of the present invention, and the particularly significant features that are thus proposed, are that the conditions required for offering, within a valve arrangement, in the form of a multiseated valve, specially adapted as a mixing valve, that offers at least one valve seating with at least two openings and a valve gate with at least one opening, such an interaction with openings belonging to the valve seatings that a displacement of the valve gate relative to the valve seating or a displacement of the valve seating relative to the valve gate can take place, have in this way been created, and in this way to make it possible to create the conditions required for controlling and adjusting with high precision a chosen mixing ratio and/or a chosen rate of flow through a displacement of a plane valve seating and a plane valve gate relative to each other.

The advantage also lies in that it is possible for the valve arrangement according to the present invention to acquire a particularly appropriate use as a multiseated valve for the mixture of a number of compressible media in a lung ventilator unit, in particular a lung ventilator unit in association with an intravenous treatment, assigned to anaesthesia, where the compressible media have principally been given the form of oxygen and air.

The possible primary characteristics of a valve arrangement according to the present invention are specified in the characterising part of the attached patent claim 1 and the possible primary characteristics of a valve arrangement when it is used as a multiseated mixing valve for the mixing of a number of compressible media in a lung ventilator unit are specified in the characterising part of the attached patent claim 6.

### SHORT DESCRIPTION OF THE DRAWINGS

A currently suggested embodiment demonstrating particular significant characteristics that are associated with the present invention will now be described in more detail for the purposes of exemplification with reference to the attached drawings in which:

FIG. 1 shows the principles for a mixing of two compressible media for use in a lung ventilator unit, where the desired mixture of oxygen and air takes place each through one regulator, and where the rate of flow to a patient is controlled by a separate regulator located downstream.

FIG. 2 shows a plan view of a valve gate with its associated interface and provided with one opening,

FIG. 3 shows a plan view of a valve seating with its associated interface and provided with two openings, one for each medium,

FIG. 4 illustrates an interaction between the interfaces according to FIGS. 2 and 3 and where the interface according to FIG. 2 can be regarded as being placed over the interface according to FIG. 3,

FIG. 5 shows in a first perspective view the valve arrangement according to the invention,

FIG. 6 shows in a second perspective view the valve arrangement according to the invention, and

FIG. 7 shows a section through the valve arrangement, according to FIGS. 5 and 6, in order thereby to make clear the design of the valve arrangement.

### DESCRIPTION OF THE EMBODIMENT SUGGESTED HERE

It should initially be made clear that we have chosen terms and a special terminology in the subsequent description of an embodiment suggested at the present time that demonstrates the significant characteristics associated with the invention and that is described in the figures shown in the attached drawings, in order, primarily, to make clear the innovative concept of the invention.

It should, however, be taken into consideration that the expressions chosen here are not to be seen as limiting to only the terms chosen and used here, but it is rather to be understood that each such chosen term is to be interpreted such that it additionally covers all technical equivalents that act in the same manner, or in essentially the same manner, in order in this way to achieve the same intention and/or technical result, or essentially the same intention and/or technical result.

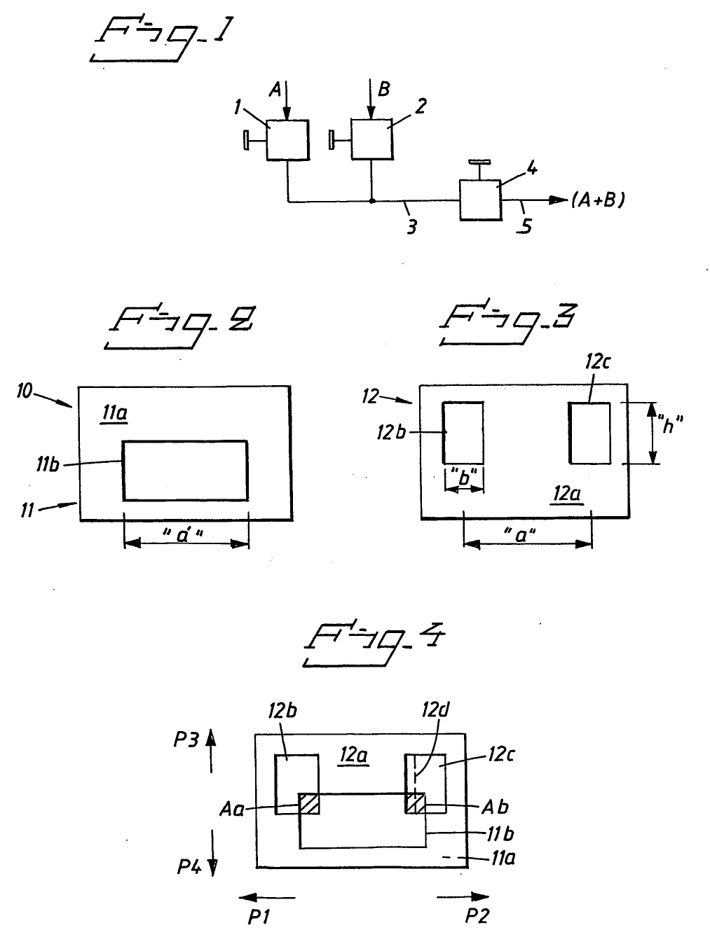

With reference to FIG. 1, a previously known arrangement is shown that makes it possible thereby to control the desired mixing ratio between two gaseous media that are used, and also to offer control of the rate of flow.

The application with two compressible media, where the first “A” is constituted by oxygen and the second “B” is constituted by air, shows the use of a regulator, in the form of a valve 1, for oxygen and a regulator, in the form of a valve 2, for air and which regulators 1, 2 are mutually adapted to receive thorough a line 3 a mixture of air and oxygen with a predetermined mixing ratio.

A regulator, in the form of a valve 4, is adapted such that it is possible through this to offer regulation of a chosen rate of flow (A+B) through a line 5, and which rate of flow then is to be administered to a patient (not shown in the figure) who is the subject of an intravenous treatment, assigned to anaesthesia.

The construction of the valves 1 and 3 within one casing is also previously known, and measures have also been taken in attempts to co-construct the valves 1, 2 and 4 within one and the same casing.

The present invention is based upon using a relative displacement between a valve gate and a valve seating, or vice versa, and where a valve gate 11 (shown in FIG. 2) for a valve arrangement 10 offers a glide surface or an interface 11a, and in which interface an opening 11b has been formed.

A valve seating 12 (shown in FIG. 3) also offers a glide surface or an interface 12a, in which interface a first opening 12b and a second opening 12c have been formed, each representing one compressible medium.

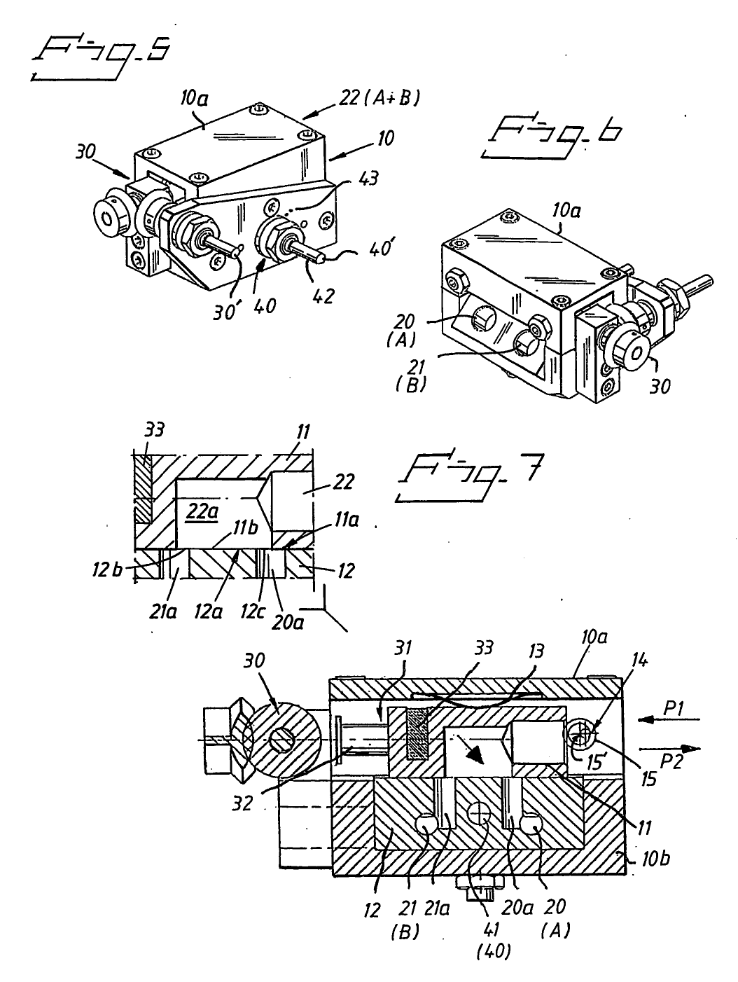

FIG. 4 intends to illustrate one embodiment in which the interface 12a is to be covered by the interface 11a and where a displacement of interface 11a relative to interface 12a can displace its opening 11b in order to open and cover the openings 12b and 12c to differing degrees.

FIG. 4 illustrates a position in which the opening 11b is centrally placed relative to the openings 12b and 12c and it illustrates that the opening 11b in the interaction with the opening 12b that is illustrated offers an open surface section “Aa” while the opening 12c in a corresponding manner offers an open surface section “Ab”.

It is now clear that in the case in which the opening 11b in the valve gate 11 is displaced to the left, in the direction of arrow P1 in FIG. 4, this will mean an increase in the surface area “Aa” at the expense of the surface area “Ab” and a changed mixing ratio arises between the used media “A” and “B” in this way.

A displacement in the direction of arrow P2 will mean, in a corresponding manner, an increase in the opened surface area “Ab” at the expense of a reduction in the surface area “Aa” and a changed mixing ratio is obtained in this way.

A displacement of the interface 11a in the direction of the arrow P3 means that the surface areas “Aa” and “Ab” increase to a equal degrees and thus the rate of flow increases while the set mixing ratio is held constant.

If, on the other hand, the valve gate 11 and the interface 11a are moved in the direction of the arrow P4, this means that the open surface area “Aa” and the open surface area “Ab” decrease to a equal degrees and thus the chosen rate of flow decreases while the chosen mixing ratio remains constant.

Although the principle of mixing and that of regulating the rate of flow have been illustrated, with reference to FIG. 4, by allowing the valve gate 11 to be displaced along perpendicular co-ordinates relative to a fixed valve seating, it should be clear that it would be possible to offer relative motion between the valve gate and the valve seating in another manner.

Other patterns of relative displacement and other forms for the openings (11b; 12b, 12c) fall within the scope of the patent, whereby the conditions can be created that are necessary for obtaining the relationships that ensure that the control of the mixing ratio and/or the control of the rate of flow will not be a linear function of a chosen displacement motion.

A valve arrangement, in the form of a multiseated mixing valve for the mixing of a number of compressible media in a lung ventilator unit will advantageously require linearity in the displacement motions.

FIG. 4 further shows that it would be possible for the valve seating 12 to be fixed relative to a casing and that only the valve gate 11 to be assigned the displacement motion that is required in order to obtain a chosen mixing ratio and/or a chosen rate of flow. It would be possible for the valve gate 11 to be fixed, and only the valve seating 12 arranged to be mobile.

The present invention, however, proposes an embodiment in which the valve gate 11 is arranged to be mobile in one direction relative to the valve seating, while the valve seating is arranged to be mobile in a first and a second direction, relative to the valve gate, and it is possible in this way for the chosen mixing ratio and a chosen rate of flow to be controlled each via one displacement motion.

With reference to FIGS. 5 and 6, two perspective views are shown of the valve arrangement 10 according to the present invention and this valve arrangement offers at least two inlets 20 and 21, intended for the media “A” and “B”, and at least one outlet 22.

A chosen mixture of the said media will flow out through the outlet 22, where it is fully possible that the mixing ratio could be chosen from only medium “A”, though an equal mixture (“A”+“B”) and over to only the second medium “B”, depending on the position of the valve gate 11 relative to the valve seating 12, in the direction of the arrows P1 and P2.

The said inlets 20, 21 in FIG. 7 are adapted to interact each via a section of channel 20a, 21a with the associated openings 12b and 12c in the valve seating 12.

The said outlet 22 is adapted to interact via a section of channel 22a with an associated opening 11b in the valve gate 11.

The invention here uses a first means 30 to control the motion of the valve gate 11 relative to the motion of the valve seating 12 at least in such a manner that at least one chosen mixing ratio and/or rate of flow can be adjusted.

In particular, the present invention proposes that a glide surface and/or an interface between the valve seating 12 and the valve gate 11, where the interface has been given the reference numerals 11a, 12a, is chosen to be plane.

However, there is nothing to prevent allowing the interfaces between the valve seating and the valve gate in any case to be chosen to be essentially plane, while on the other hand the interfaces 11a and 12a must offer an airtight, or at least essentially airtight, connection while they interact, via a layer of lubricant acting within the interface. This is not shown in detail in FIG. 7.

The present invention proposes the use of a further means 40.

This means 40 is adapted such that it can control the motion of the valve seating 12 relative to the valve gate 11 and in this way at least one chosen rate of flow can be adjusted by using the directions of displacement P3, P4.

The said two means 30, 40 are, in order to be able to control individually the forwards and backwards motion of the valve gate 11 and the forwards and backwards motion of the valve seating 12, designed to comprise two separate devices 31, 41, each controlling one of the motions.

A first device 31 is adapted for a first direction of motion P1 and P2, and a second device 41 is adapted for a second direction of motion P3 and P4.

A first direction of motion P1 and P2 is adapted to exclusively, or at least to a dominating degree, regulate the mixing ratio between the media while the second direction of motion is adapted to exclusively, or at least to a dominating degree, be able to regulate the rate of flow passing through the outlet 22.

The first device 31, which controls the motion of the valve gate 11, is adapted to endow the valve gate 11 via a rotational motion around an axis 32 with a translation or displacement motion oriented along the interface 11a through the axle 32 offering a thread interacting with a corresponding thread in a unit 33 serving as a nut.

The second device 41, which controls the motion of the valve seating 11, is adapted to endow the valve seating 12 via a rotational motion around an axis 42 with a translation or displacement motion oriented along the interface 12a where the direction of motion and/or speed of displacement of the latter is chosen to be distinct from the direction of motion and/or the speed of displacement via the first device.

The axis 42 interacts in a rotational manner with the valve seating 12 while remaining fixed, and it offers a threaded section, which interacts with a unit 43 that serves as a nut, in order in this way to endow the valve seating with the desired direction of motion in the direction of the arrow P3 or P4.

The embodiment according to FIG. 7 illustrates that the directions of motion have here been chosen to be straight and distinct from each other, preferably at an angle of approximately 90°.

When the valve arrangement is to be used as a multiseated mixing valve for the mixing of a number of compressible media in a lung ventilator unit, in particular a lung ventilator unit in association with an intravenous treatment, assigned to anaesthesia, it is suggested according to the invention that the said media “A”, “B” are supplied to the said inlets 20, 21 at the same pressure, or at least at essentially the same pressure.

Thus the possibility is offered for an accurate regulation of the rate of flow to be carried out via the further means 40 and the device 41 associated with it without influencing the mixing ratio.

An accurate control of the mixing ratio can be carried out via the said means 30 and the device 31 associated with it, without influencing the rate of flow.

In the situation in which the media used “A”, “B” have different densities, the invention offers the possibility of compensating for this by selecting for the higher density an area of opening “Ab” that is smaller than the area of opening “Aa” in FIG. 4.

This compensation can occur though displacing a section of wall for the opening 12c somewhat to the right in FIG. 4 according to the dashed line 12d.

The gearing ratio between a chosen rotational motion, for example of the axle 32, and a forwards and backwards displacement motion, such as for the gate 11, can advantageously be adapted to correspond to the relationship: one revolution=0.5-2.0 mm, for example 0.6- 1.2 mm.

In particular, the mixing ratio can be chosen more accurately by using a smaller gradient for the screw arrangement than the control of the rate of flow, which uses a greater gradient for the screw arrangement than is used there.

The shaped openings 12b, 12c in the valve seating 12 are according to FIG. 3 arranged side-by-side at a separation of “a” that corresponds to a chosen length “a” for the opening 11b in the valve gate 11.

The openings in the valve seating 12 are in the interface 12a according to the embodiment chosen to be right-angled and rectangular, with a width “b” corresponding to the total displacement motion for the chosen limiting values for the chosen mixing ratio, and with a length or height “h” corresponding to the total displacement motion for the chosen limiting values of the chosen regulation of rate of flow.

In particular, the openings 12b, 12c in the valve seating 12 within the interface 12a can be chosen with a narrowing or a increasing width (not shown in the drawing) within the said distance “h” related to length.

Alternatively, the opening 11b in the valve gate 11 within the interface 11a, and at least within the openings 12b, 12c in the valve seating 12, can be chosen with a narrowing or increasing width (not shown in the drawing).

With reference again to FIG. 7, the valve gate 11 is there shown pressed with the aid of a bent and flat spring unit 13 against the valve seating 12, such that the previously specified interfaces 11a, 12a are in this way formed.

The first directions of motion P1, P2 are offered through a turning motion at an angle, whereby the two displacement motions can take place via one each of two parallel axes of rotation 40′, 30′.

The valve seating 11 is arranged such that it can be displaced in a forwards and backwards motion within a first casing section 10a while the valve gate 12 is arranged such that it can be displaced forwards and backwards within a box-shaped second casing section 10b.

The rotational motion for the axle 32 interacts with a nut piece 33, which is fixed, but interacts in a somewhat mobile manner with the valve gate 11.

A chosen ratio between an open area “Aa” for the one opening 12b in the valve seating and the opening 11b in the valve gate 11, and an open surface “Ab” for the second opening 12c in the valve seating 12 and the opening 11b in the valve gate 11 is adapted to offer a chosen mixing ratio.

The sum of the open surface area “Aa” for the one opening 12b in the valve seating and the opening 11b in the valve gate 11 and an open surface area “Ab” in the second opening 12c in the valve seating 12 and the opening 11b in the valve gate 11 is adapted to offer a chosen rate of flow.

A stop 14 that can be regulated is adapted to limit the motion of the valve gate 11 to the right in FIG. 7 and where the controlling effect arises in that a peg 14 is attached at the side of the axis or rotation 15′ for an axle 15 that can be turned.

Naturally, the invention is not limited to the embodiments described above as examples. The invention can be subject to modifications within the scope of the innovative concept as illustrated by the attached patent claims.

Special attention should be paid to the fact that each accessory shown can be combined with each other accessory shown within the scope in order to be able to achieve the desired technical function.

1. A valve arrangement (10) with at least two inlets (20, 21), adapted each for a medium (A, B), and at least one outlet (22), through which a chosen mixture of the said media can pass, the said inlets (20, 21) being furthermore adapted to interact each via a section (20a, 21a) of channel with associated openings (12c, 12b) in a valve seating (12) while the said outlet (22) is adapted to interact via a section (22a) of channel with an associated opening (22) in a valve gate (11), whereby a first means (30) is available in order to control at least the motion of the valve gate (11) relative to the valve seating (12) or vice versa in such a manner that at least one chosen mixing ratio and/or one chosen rate of flow can be regulated, characterised in that a glide surface and/or an interface (11a, 12a) between the valve seating (12) and the valve gate (11) is chosen to be plane, or at least essentially plane, that a further means (40) is available in order to control the motion of the valve gate (11) relative to the valve seating (12) or vice versa in such a manner that the said two means (30, 40) in order to control the motion of the valve gate (11) and/or the motion of the valve seating (12) or vice versa, are formed such that they comprise two distinct devices (31, 41) each controlling one motion.

2. The valve arrangement according to claim 1, characterised in that a first device (31) is adapted for a first direction of motion and a second device (41) is adapted for a second direction of motion, and that the first direction of motion is adapted to exclusively, or at least to a dominant degree, regulate the mixing ratio between the media, while the second direction of motion is adapted to exclusively, or at least to a dominant degree, regulate the rate of flow of the mixture passing through the outlet.

3. The valve arrangement according to claim 1, characterised in that the first device (31), which controls the motion of the valve gate, is adapted to endow via a rotational motion a translation or displacement motion oriented parallel to the interface (11a) to the valve gate (11).

4. The valve arrangement according to claim 1, characterised in that the second device (41), which controls the motion of the valve seating, is adapted to endow via a rotational motion a translation or displacement motion oriented parallel to the interface (12a) to the valve seating (12), where the direction of motion of the latter and/or its speed of displacement is chosen to be different from the direction of motion and/or the speed of displacement offered by the first device.

5. The valve arrangement according to claim 3, characterised in that the directions of motion are chosen to be straight and distinct from each other, preferably at an angle of approximately 90°.

6. The valve arrangement according to claim 1, used as a multiseated mixing valve for the mixing of a number of compressible media in a lung ventilator unit, in particular a lung ventilator unit in association with an intravenous treatment, assigned to anaesthesia, characterised in that the said media are supplied to the said inlets at the same pressure, or at essentially the same pressure, whereby a regulation of the rate of flow, via the further means (40) and its associated device (41) can be carried out without influencing the mixing ratio, or a regulation of the mixing ration, via the said means (30) and its associated device (31), can be carried out without influencing the rate of flow.

7. The valve arrangement according to claim 1, characterised in that the gear ratio between the chosen rotational motion/displacement motion is adapted to correspond to: one revolution=0.5-2.0 mm, for example 0.6-1.2 mm.

8. The valve arrangement according to claim 1, characterised in that the openings formed in the valve seating are arranged side-by-side and at a distance from each other that corresponds to a chosen length of the opening in the valve gate.

9. The valve arrangement according to claim 8, characterised in that the openings in the valve seating are chosen to be right-angled in the interface, with a width that corresponds to the total displacement motion for the chosen limiting values of the chosen mixing ratio and with a length that corresponds to the total displacement motion for the chosen limiting values for the chosen regulation of rate of flow.

10. The valve arrangement according to claim 8 characterised in that the openings in the valve seating are, within the interface, chosen with a narrowing or with a widening width within the said distance related to length.

11. The valve arrangement according to claim 8, characterised in that the opening in the valve gate is, within the interface and at least within the openings in the valve seating, chosen with a narrowing or with a widening width.

12. The valve arrangement according to claim 1, characterised in that the valve gate is pressed against the valve seating with the aid of a spring unit.

13. The valve arrangement according to claim 1, characterised in that the first direction of motion is offered via a rotational motion at an angle, whereby the two displacement motions can occur each via one of two axes of rotation arranged parallel to each other.

14. The valve arrangement according to claim 1, characterised in that the valve seating is arranged to be displaceable forwards and backwards in a box-shaped first casing section.

15. The valve arrangement according to claim 1, characterised in that the valve gate is arranged to be displaceable backwards and forwards in a box-shaped second casing section.

16. The valve arrangement according to claim 6, characterised in that the rotational motion interacts with a nut, but interacting in a somewhat flexible manner with the valve gate.

17. The valve arrangement according to claim 1, characterised in that a chosen ratio between the open surface area for one opening in the valve seating and the opening in the valve gate and an open surface area for the second opening in the valve seating and the opening in the valve gate is adapted to offer a chosen mixing ratio.

18. The valve arrangement according to claim 1, characterised in that the sum of the open surface area for the one opening in the valve seating and the opening in the valve gate and an open surface area for the second opening in the valve seating and the opening in the valve gate is adapted in order to offer a chosen rate of flow.
